(19) United States
(12) Patent Application Publication
(10) Pub. No.: US 2023/0036959 A1
(43) Pub. Date: Feb. 2, 2023

(54) BILE TOLERANT PROBIOTICS TO INHIBIT ENTERIC PATHOGENS

(71) Applicant: SOUTH DAKOTA BOARD OF REGENTS TECHNOLOGY TRANSFER OFFICE, Brookings, SD (US)

(72) Inventor: Joy Scaria, Brookings, SD (US)

(21) Appl. No.: 17/790,934

(22) PCT Filed: Jan. 4, 2021
(86) PCT No.: PCT/US2021/012106

§ 371 Date: Jul. 5, 2022

Related U.S. Application Data
(60) Provisional application No. 62/957,012, filed on Jan. 3, 2020.

(51) Int. Cl. C12Q 1/04; C12Q 1/18; A61K 35/742; C12N 1/20

(52) U.S. Cl. CPC C12Q 1/045; A61K 35/742; C12N 1/20; C12Q 1/18

(57) ABSTRACT

A composition and method for identifying a microbial composition that inhibits colonization of an enteric pathogen in an animal is disclosed. The method includes removing a microbial sample from a digestive tract of at least one healthy individual, culturing the microbial sample, isolating one or more microbial species within the microbial sample, and identifying one or more isolated microbial species. The method further includes determining one or more bile-tolerant properties of the one or more isolated microbial species, and creating one or more microbial compositions of the one or more isolated microbial species that are bile-tolerant. The method further includes determining an ability of the one or more microbial compositions to inhibit growth of an enteric pathogen in at least one of an assay, and identifying one or more microbial compositions from the one or more microbial compositions capable of inhibiting growth of enteric pathogens in an animal.

### CROSS-REFERENCE TO RELATED APPLICATIONS

The present application claims priority to U.S. Provisional Application Serial No. 62/957,012, filed Jan. 3, 2020, which is incorporated herein by reference in the entirety.

### TECHNICAL FIELD

The present invention generally relates to probiotics, and in particular to the use of bile tolerant probiotics to prevent disease in humans.

### BACKGROUND

Normal bacterial flora in the human gut can eliminate pathogens through various immunological responses under normal conditions in which the host intestine is protected commensal bacteria in a phenomenon described as colonization resistance (CR). Protective commensal bacteria can indirectly control pathogen invasion by enhancing host immunity through several mechanisms, including competing for niches and nutrients, metabolic exclusion by producing short chain fatty acids (SCFAs), and production of antimicrobial peptides. Recently, bile conversion has emerged as a promising direct mechanism by which the bile converting bacteria in the gut inhibit pathogen especially against Clostridioides difficile infection (CDI).

Clostridioides difficile is a Gram-positive, spore-forming anaerobe and a major causative agent for antibiotic-induced diarrhea in hospitalized patients. Dysbiotic gut conditions following antibiotic treatment have been attributed as a major predisposing factor for C. difficile infections. The primary treatment for CDI is vancomycin and fidaxomicin, however, 15-30% of CDI-treated patients’ relapse. Fecal microbiota transplantation (FMT) has been highly effective for treatment of CDI, with an approximately 90% success rate. However, the use of FMT has been controversial due variable bacterial community of the fecal transplant, differences in recipient genotype and possible transfer of pathogens Recently, microbial metabolism of bile acids has been recognized as one of the major direct mechanisms against CDI in the intestine.

Bile, containing primary bile salts, is synthesized in the liver and is secreted into the proximal small intestine from the hepatobiliary tract, where it eventually reaches bacteria in the large intestine, which convert the primary bile salts to secondary bile salts through deconjugation from an amino acid moiety and dihydroxylation, producing deoxycholate (DCA) and lithocholate. High accumulation of DCA is highly toxic to vegetative C. difficile cells and less toxic to many commensal bacterial species. However, high DCA accumulation also damages colon epithelial cells, which may lead to colon cancer. As such, there is a desire to determine and utilize a standardized bile-tolerant probiotic treatment for patients with CDI and CDI like diseases that does not damage colon epithelial cells.

### SUMMARY

A method for identifying a microbial composition that inhibits colonization of an enteric pathogen in an animal is disclosed. In some embodiments, the method includes removing a microbial sample from a digestive tract of at least one healthy individual. In some embodiments, the method includes culturing the microbial sample. In some embodiments, the method includes isolating one or more microbial species within the cultured microbial sample. In some embodiments, the method includes identifying the one or more isolated microbial species. In some embodiments, the method includes determining one or more bile-tolerant properties of the one or more isolated microbial species. In some embodiments, the method includes creating one or more microbial compositions of the one or more isolated microbial species that are bile-tolerant. In some embodiments, the method includes determining an ability of the one or more microbial compositions to inhibit growth of an enteric pathogen in at least one of an in vitro or an in vivo assay. In some embodiments, the method includes identifying at least one of the one or more microbial composition from the one or more microbial compositions capable of inhibiting growth of enteric pathogens in an animal.

A microbial composition and methods for identifying and administrating a microbial composition that inhibits colonization of an enteric pathogen in an animal is disclosed that is prepared by a process. In some embodiments, the process includes removing a microbial sample from a digestive tract of at least one healthy individual. In some embodiments, the process includes culturing the microbial sample. In some embodiments, the process includes isolating a microbial species within the cultured microbial sample. In some embodiments, the process includes identifying one or more isolated microbial species. In some embodiments, the process includes determining one or more bile-tolerant properties of the one or more isolated microbial species. In some embodiments, the process includes creating one or more microbial compositions of the one or more isolated microbial species that are bile-tolerant. In some embodiments, the process includes determining an ability of the one or more microbial compositions to inhibit growth of an enteric pathogen in at least one of an in vitro or an in vivo assay. In some embodiments, the process includes identifying at least one of the one or more microbial composition capable of inhibiting growth of enteric pathogens in an animal. In some embodiments, the process includes fashioning at least one of the one or more microbial composition into a form capable of enteric administration.

A method of administering a bile-tolerant microbial composition that inhibits colonization of an enteric pathogen in animals is disclosed. In some embodiments, the method includes identifying an animal with an at least one of an active enteric disease or risk of enteric disease, administering to the animal a microbial composition comprised of a mixture of at least one of a bile-tolerant microbial species.

### BRIEF DESCRIPTION OF THE DRAWINGS

The numerous advantages of the disclosure may be better understood by those skilled in the art by reference to the accompanying figures in which:

FIG. 1 is a flow diagram illustrating a method for identifying a bile-tolerant microbial composition that inhibits the colonization of enteric infections in an animal, in accordance with one or more embodiments of the present disclosure;

FIG. 2 is a flow diagram illustrating a method of administering a microbial composition that inhibits colonization of an enteric pathogen in an animal, in accordance with one or more embodiments of the present disclosure;

FIG. 3A is a graph illustrating high bile tolerance of selected bacterial species, in accordance with one or more embodiments of the present disclosure;

FIG. 3B is a graph illustrating low bile tolerance of selected bacterial species, in accordance with one or more embodiments of the present disclosure;

FIG. 3C is a graph illustrating the ability of high bile tolerant and low bile tolerant bacterial species to grow in the presence of bile, in accordance with one or more embodiments of the present disclosure;

FIG. 4A is a chart illustrating a detailed timeline for testing bile tolerance of bacteria in a continuous flow model, in accordance with one or more embodiments of the present disclosure;

FIG. 4B is a graph illustrating the inhibition of C. difficile by bacterial blends tolerant to high and low concentrations of bile, in accordance with one or more embodiments of the present disclosure;

FIG. 4C is a graph illustrating the inhibition of C. difficile by bacterial blends tolerant to high and low concentrations of bile in the absence of bile, in accordance with one or more embodiments of the present disclosure;

FIG. 4D is a graph illustrating the inhibition of C. difficile by bacterial blends tolerant to high and low concentrations of bile in the presence of bile, in accordance with one or more embodiments of the present disclosure;

FIG. 4E is a graph illustrating the inhibition of C. difficile by bacterial blends tolerant to high concentrations of bile in the presence or absence of bile, in accordance with one or more embodiments of the present disclosure;

FIG. 4F is a graph illustrating the inhibition of C. difficile by bacterial blends tolerant to low concentrations of bile in the presence or absence of bile, in accordance with one or more embodiments of the present disclosure;

FIG. 5A is a graph illustrating the inhibition efficiency of CD R20291 by HBT10 and LBT10 consortiums, in accordance with one or more embodiments of the present disclosure;

FIG. 5B is a graph illustrating the effect of HBT10 on CD CFU counts, in accordance with one or more embodiments of the present disclosure;

FIG. 5C is a graph illustrating the effect of LBT10 on CD CFU counts, in accordance with one or more embodiments of the present disclosure;

FIG. 5D is a graph illustrating the effect of HBT10 and bile on CD CFU counts, in accordance with one or more embodiments of the present disclosure;

FIG. 5E is a graph illustrating the effect of LBT10 and bile on CD CFU counts, in accordance with one or more embodiments of the present disclosure;

FIG. 6A is a graph illustrating the effect of HBT10/CD or LBT10/CD cocultures on acetate levels, in accordance with one or more embodiments of the present disclosure;

FIG. 6B is a graph illustrating the effect of HBT10/CD or LBT10/CD cocultures on propionate levels, in accordance with one or more embodiments of the present disclosure;

FIG. 6C is a graph illustrating the effect of HBT10/CD or LBT10/CD cocultures on butyrate levels, in accordance with one or more embodiments of the present disclosure.

### DETAILED DESCRIPTION OF THE INVENTION

Reference will now be made in detail to the subject matter disclosed, which is illustrated in the accompanying drawings.

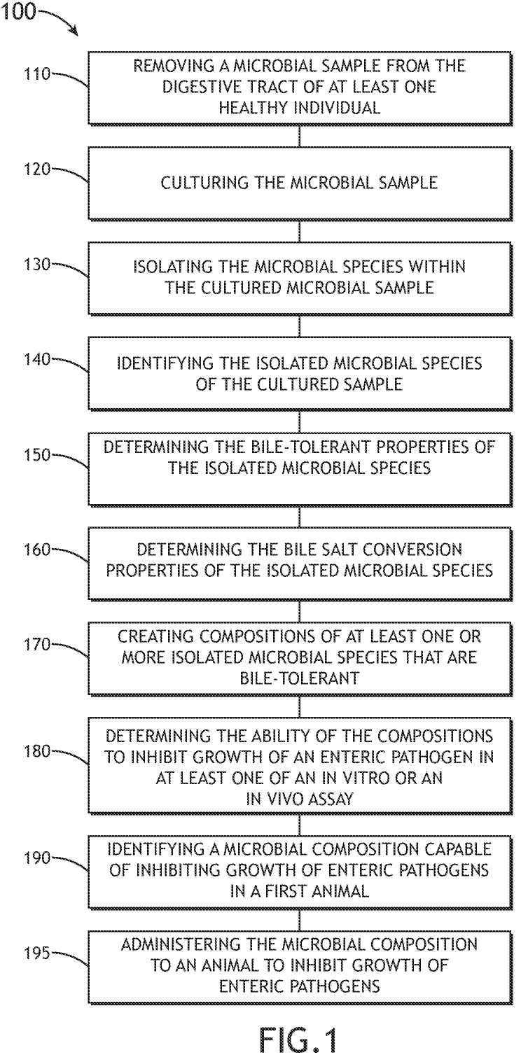

FIGS. 1-6 generally illustrate a composition and methods for inhibiting the colonization of enteric infections in animals utilizing bile-tolerant probiotics, in accordance with one or more embodiments of the present disclosure. The composition augments the ability of the gut of an animal to resist enteric infections through colonization resistance.

FIG. 1 illustrates a method 100 for identifying a bile-tolerant microbial composition that inhibits the colonization of enteric infections in an animal. In some embodiments, the first animal is a human. In other embodiments, the animal is a domesticated animal including, but not limited to, dogs, cats, cattle, pigs, poultry, or fish.

In embodiments, the enteric pathogen may include any type of enteric pathogens known to cause an enteric disease, including, but not limited to, viruses, bacteria (e.g., from the phyla Firmicutes, Bacteroidetes, Actinobacteria, and Proteobacteria), fungi, protists, archaea, and multicellular parasites. For example, the enteric pathogen may be C. difficile.

In embodiments, the method also includes a bile-tolerant microbial composition that inhibits colonization of enteric pathogens. The microbial composition may take the form of any type of composition commonly used for entry into the digestive tract of an animal. For example, the microbial composition may be configured as a powder that is dissolved in liquid for the animal to drink. In another example, the microbial composition may also be configured as a capsule or pill that is ingested. In another example, the microbial composition may be configured as a granular or pelleted form (e.g., a granule or pellet) that is ingested. For instance, the granular form may be mixed in with other feed components (i.e., a sprinkle formulation). In another example, the microbial composition may be configured as a liquid to be taken orally. For instance, the microbial composition may be configured as a liquid that is sprayed on livestock feed. In another example, the microbial composition may be configured as a semi-solid liquid (i.e., having the consistency of yogurt). In another example, microbial composition may be configured as a suppository or other type of formulation for use rectally. Alternatively, the microbial composition may be a liquid that is injected into the digestive tract of an animal (e.g., inoculating an embryonic chick).

In embodiments, the microbial composition also includes an enteric coating. Any enteric coating may be used for enteric administration. For example, the enteric coating may include a polymer. Any type of coating polymer may be utilized including but not limited to shellac (e.g., aleurtic acid esters), cellulose acetate phthalate, poly(methacrylic acid-co-methyl methacrylate), cellulose acetate trimellitate, poly(vinyl acetate phthalate), and hydroxypropyl methylcellulose phthalate. In another example, the microbial

In embodiments, the microbial composition includes a binder. Any type of binder may be used. For example, the binder may be configured for capsule formulation or pill formulation binder that includes but is not limited to gelatin, cellulose, polyvinylpyrrolidone, starch, sucrose, and polyethylene glycol. In another example, the binder may be configured for pellet formation that includes but is not limited to lignin-based binders, hemi-cellulose binders, and mineral binders (i.e., clays).

In embodiments, the method 100 includes step 110 of removing a microbial sample from the digestive tract of at least one healthy individual. For example, a microbial sample from a single healthy individual may be used in the method. In another example, pooled samples from ten healthy individuals may be used in the method. In still another example pooled samples from 100 healthy individuals may be used in the method.

In embodiments, the method 100 further includes the step 120 of culturing the microbial sample. The culture medium used for culturing the microbial sample may be any type of growth media known in the art for growing microbes, including, LB broth, blood agar, chocolate agar, brain heart infusion media, and the like. In some embodiments, the culture media is a modified brain heart infusion media (e.g., mBHI).

Culturing the microbial sample also involves environmental conditions (e.g., temperature, gas content). In embodiments, the temperature for culturing the microbial sample is the temperature of the gut of the animal (e.g., 35° C. to 42° C. In some embodiments, the temperature of the culture is 37° C. Alternatively, the temperature of the culture is room temperature (e.g., 20° C. to 25° C.). The culture may be grown in an anaerobic or low oxygen environment. Alternatively, the culture may be grown in an open atmosphere environment.

In some embodiments, an iterative antibiotic supplementation is used to suppress bacteria that dominates the culture medium. The antibiotics used in the iterative antibiotic supplementation include any antibiotics known to suppress the growth of bacteria or other biological entities, including, but not limited to, gentamycin, kanamycin, neomycin, sulfamethoxazole, clindamycin, ampicillin, erythromycin, vancomycin, chloramphenicol, metronidazole, colistin, and the like. In embodiments, any mixture of antibiotics may be used in the iterative antibiotic supplementation. The iterative antibiotic supplementation may also include a heat treatment step. In some embodiments, no iterative antibiotic supplementation is used.

Method 100 further includes step 130 of isolating the microbial species in the cultured microbial sample. Isolating microbial species may involve plating of the cultured microbial sample, resulting in the growth of individual colonies. Alternatively, the microbial species may be isolated through serial dilutions of the microbial sample.

In embodiments, the method 100 further includes the step 140 of identifying the microbial species within the cultured microbial sample. Identification of microbial species may include any method known in the art for identifying microbes, including genomic, proteomic, biochemical, and the like. Genomic methods for identifying microbial species include any methods known in the art for identifying microbial species, including, but not limited to, ribosomal RNA sequencing (e.g., 16S rRNA, 18S rRNA 28S rRNA, etc.), gene specific sequencing (e.g., rpoB, tuf, gyrA, gyrB, sodA), loop-mediated isothermal amplification assay, and microarray. Ribosomal RNA and gene specific sequences may be generated using any sequencing technology in the art, including traditional slab sequencing, Illumina sequencing, 454 pyrosequencing, and the like. For example, whole genomes of enteric pathogens may be sequenced using the Illumina MiSeq platform.

Proteomic methods for identifying microbes include any proteomic methods capable of identifying of identifying microbes, including, but not limited to, MALDI-TOF MS, tandem mass spectrometry, and peptide sequencing. Biochemical methods may include the use of specific stains (e.g., Gram, acid-fast), antibody detection, and probe hybridization (e.g., FISH).

In embodiments, the method 100 further includes step 150 of determining the bile-tolerant properties of the isolated microbial species. Microbial species with high tolerance to bile and bile products (e.g., DCA and lithocholate) may prevent pathogens like C. difficile from colonizing the gut. Methods to test microbial strains for bile-tolerance include any bile-tolerance tests known in the art, including but not limited to bile-supplementation of growth media.

The method 100 further includes step 160 of determining the bile salt conversion properties of the isolated microbial species. As mentioned previously, commensal microbes that generate high concentrations of DCA kill pathogens, but may injure intestinal tissue. Microbes that generate moderate or low DCA levels may still produce high enough DCA to kill pathogens while leaving intestinal cells and tissue unharmed. Methods to determine the bile salt conversion properties of microbial species include any methods known in the art including but not limited to determining if the microbes have bile acid inducible (BAI) genes in their genome (e.g., including but not limited to baiA1/3, baiA2, baiCD, baiE, baiF, baiG, baiH, bail and baiJKL genes). Methods that directly measure conversion of bile salts by microbial strains may also be utilized. Once the bile-salt conversion properties of the microbial strains and species are determined, the microbial strains may be selected for further use in a microbial composition based on the conversion properties. In some embodiments, the method 100 does not include step 160 of determining the bile salt conversion properties of the isolated microbial species.

The method 100 further includes the step 170 of creating compositions of at least one or more bile-tolerant microbial species. The selection of an isolated microbial species in a microbial composition may depend on the ability of the microbial species to inhibit growth of the enteric pathogen in vitro or in vivo. The selection of microbial species may also depend on the previously known abilities of mixtures of various microbial species to inhibit enteric pathogens.

In embodiments, the method 100 includes the step 180 of determining the ability of the compositions to inhibit growth of an enteric pathogen in at least one of an in vitro or in vivo assay. In vitro determination of microbial compositions includes co-culture assays in static or continuous flow systems, where both the microbial composition and the enteric pathogen are cultured together in liquid media in the presence or absence of bile salts. After an incubation period, the broth may then be serially diluted and plated on agar plates, and the number of colony forming units (CFU) are assessed.

In vivo determination of microbial composition includes testing the ability of the microbial composition to inhibit growth of enteric pathogens in a model animal. The animal used for testing microbial compositions may include any model animal that is relevant for testing. For example, for identifying microbial compositions effective in humans, the model animal may be a pig. In this in vivo test, the pigs are fed both the microbial composition and the enteric pathogen. After an incubation period, the pig is examined for the presence of the enteric pathogen and damage caused by the enteric pathogen. In the in vivo test, the animal may be gnotobiotic, having no flora within the digestive tract (e.g., an animal previously treated with antibiotics or a recently born/hatched animal). Alternatively, an animal possessing flora within the digestive tract may be used.

In some embodiments, the method 100 includes the step 190 of identifying a microbial composition capable of inhibiting growth of enteric pathogens in an animal. The microbial composition may include any microorganism that has been identified to inhibit growth of an enteric pathogen. Finally, in some embodiments, the method 100 includes the step 195 of administering the microbial composition to an animal to inhibit growth of enteric pathogens. In some embodiments, the microbial composition may include an antibiotic. For example, microbial composition may include an antibiotic shown via the iterative antibiotic supplementation to suppress dominating bacterial strains, and keep a stochastic relationship between the species of the strains within the microbial composition.

It should be understood that the microbial composition produced by the method 100 is not a microbial composition that is found in nature. The microbiome of animals generally includes a mix of bile-tolerant and bile-intolerant microbes, wherein the microbial composition herein generally includes only bile-tolerant microbes. Furthermore, the microbial composition produced by the method 100 includes a fixed set of microbial species (e.g., less than 100 microbial species, less than 20 microbial species, less than ten microbial species, or less than five microbial species), wherein the microbial composition within the natural microbiome of an animal is typically greater than 1000 microbial species.

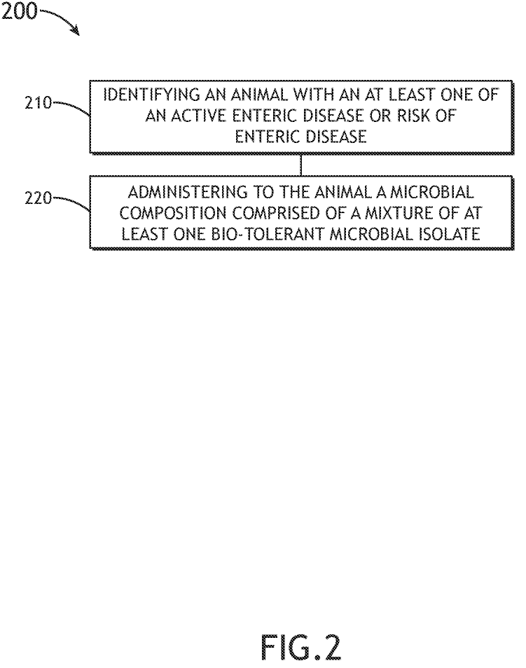

FIG. 2 illustrates a method 200 of administering a microbial composition that inhibits colonization of an enteric pathogen in animals. The method may include any step from the method 100 or any other element described herein. In some embodiments, the method 200 includes a step 210 of identifying an animal with an at least one of an active enteric disease or risk of enteric disease. For example, an ill patient at a hospital identified through testing as having a CDI. In another example, an ill patient at a hospital with a weakened immune system and a medical history of CDI may have the microbial composition administered prophylactically. Animals at risk for enteric disease include very young or very old animals, as well as animals with depressed immune systems (e.g., sick and injured animals). High-density populations of animals and animals that have live in low-diversity microbial environments (e.g., factory farms) are also risk factors for enteric disease. Alternatively, animals that are present symptoms of enteric disease may also be identified for treatment.

In some embodiments, the method further includes a step 220 of administering to the animal a microbial composition comprised of a mixture of at least one of a bile-tolerant microbial species (e.g., a bile-tolerant microbial isolate), wherein the microbial composition is administered enterically. The administration of the microbial composition may be of any route of administration commonly used in the art for administration of probiotics, including, but not limited to, enteric administration (e.g., oral, rectal). Enteric administration includes any method of delivering a therapeutic substance into the digestive tract of the subject, including, but not limited to, eating, drinking, administering through a nasogastric tube, administering through the rectum (e.g., enema, suppository) and direct injection into the digestive tract of an animal). In embodiments, the microbial composition may comprise any form known in the art capable of being administered to an animal, including, but not limited to, a pill, a tablet, a solution, a suspension, an enema, and a suppository.

Embodiments of the present disclosure are directed to a microbial composition that inhibits the colonization of an enteric pathogen in an animal. The microbial composition may be produced via any methods 100, 200 or via any step of methods 100, 200 disclosed herein. In embodiments, the microbial composition is prepared by a process that includes a number of steps. In embodiments, one step to prepare the microbial composition is to remove a microbial sample from the digestive tract of an animal. Another step to prepare the microbial composition is to culture the microbial sample. In some embodiments, the preparation of the microbial composition includes the step of isolating the microbial species within the cultured microbial sample. In embodiments, the preparation of the microbial sample further includes the identification of the isolated microbial species. The methods for identification of isolated microbial species are described herein.

In some embodiments, the preparation of the microbial composition includes the steps of determining the bile-tolerant properties of the isolated microbial species. In some embodiments, the preparation of the microbial composition includes the steps of creating compositions of at least one or more bile-tolerant microbial species. The microbial composition may also include an antibiotic (e.g., an antibiotic shown to maintain a stochastic relationship between the bacterial strains within the microbial composition). In embodiments, preparation of the microbial composition includes determining the ability of the compositions to inhibit growth of an enteric pathogen in vitro or in vitro. Methods for the testing of the microbial compositions are described herein. In some embodiments, preparation of the microbial composition includes testing on an animal to determine whether the microbial composition is capable of inhibiting the growth of enteric pathogens. Finally, in some embodiments, the preparation of the microbial composition includes the step of fashioning the microbial composition into a form capable of enteric composition (e.g., a pill, enema, or oral solution).

The following examples are intended only to further illustrate the invention and are not intended to limit the scope of the subject matter which is defined by the claims.

### Example 1

### Development of the Human Gut Microbiota Library and Whole-Genome Sequencing for Species

For the isolation of bacteria from the human gut, six intestinal samples were pooled. The pooled intestinal sample was serially diluted and was plated on modified Brain Heart Infusion agar (BHI-M) with different selective conditions. The modified Brain Heart Infusion agar (BHI-M) contained the following ingredients: 37 g/L of BHI, 5 g/L of yeast extract, 1 ml of 1 mg/mL menadione, 0.3 g L-cysteine, 1 mL of 0.25 mg/L of resazurin, 1 mL of 0.5 mg/mL hemin, 10 mL of vitamin and mineral mixture, 1.7 mL of 30 mM acetic acid, 2 mL of 8 mM propionic acid, 2 mL of 4 mM butyric acid, 100 µl of 1 mM isovaleric acid, and 1% of pectin and inulin. All cultures were performed inside an anaerobic chamber (Coy Laboratories) containing 5% CO2, 10% H2, and 85% N2 maintained at 37° C. Colonies were picked from all conditions and dilutions. Selected colonies were streaked on base BHI-M agar, and a single colony was selected for preparing stocks and species identification.

The growth of each bacterial species measured following overnight incubation in BHI-M using a spectrophotometer at OD600. Thereafter, stocks were maintained by adjusting the OD to 0.5. Aerotolerance of the strains was tested by culturing in aerobic, anaerobic and microaerophilic conditions. To this end, individual strains were first cultured overnight in BHI-M broth at 37° C. under anaerobic condition. The optical density at 600 nm (OD₆₀₀) of the cultures was adjusted to 0.5. Then, 1% of OD₆₀₀ adjusted cultures were inoculated in fresh BHI-M media in triplicates. Each replicate of cultures was then incubated under anaerobic, microaerophilic and aerobic conditions. For microaerophilic condition, a hypoxic box was used to incubate the culture. After 24 hours of incubation, the growth of individual bacteria was determined by measuring OD₆₀₀.

Genomes of 102 species from the culture library was sequenced using Illumina MiSeq platform (Illumina Inc, CA). The genomic DNA was isolated from 0.5 ml of overnight culture using E.Z.N.A.Ⓡ Bacterial DNA Kit (Omega Biotek, GA). The sequencing library was then prepared using Nextera XT Kit (Illumina Inc, CA). Libraries were sequenced using 250 base paired-end chemistry on an Illumina Miseq platform. FASTQ files were generated using Casava v1.8.2 pipeline (Illumina, Inc, CA). The FASTQ sequences were filtered for quality and sequencing adaptors with PRINSEQ. Filtered reads were then assembled de novo using Unicycler with default parameters. Each assembly result was checked for quality individually using QUAST.

### Example 2

### Individual Bile Tolerance Assay

82 bacteria that have moderate or fast growth rate were selected for the bile sensitivity assay after excluding potential pathogens and very slow growers. Each species was grown to OD600=0.5 and stocked in -80oC in 10% DMSO. Bacterial strains were tested in mBHI medium with and without bovine bile supplement (0.5 g/L of bovine bile). 20 µl of OD600=0.5 adjusted bacterial suspension was added to 180 µl of media with and without bovine bile supplement in 96 well microtiter plates and incubated anaerobically for 24 hours. OD650 was measured at 0 hours and 24 hours. Individual screening of the bacteria was performed in triplicate. Bacteria that could grow at least 40% in bovine bile supplemented media compared to normal mBHI were considered as bile resistant while others were considered as bile sensitive. Bacteria were grouped as high bile tolerant (HBT, that grow >40% in presence of bile) and low bile tolerant (LBT, that grow <40% in presence of bile) after individual screening.

To analyze the bile tolerance among the bacteria, it was important to know if these species could convert the primary bile to secondary bile. Thus, we created a custom database of bile acid-inducible (bai) genes comprising of baiA1/3, baiA2, baiCD, baiE, baiF, baiG, baiH, bail and baiJKL by accessing the genes of Clostridium scindens ATCC 35704 and Clostridium scindens VPI 12708. We searched for the presence of these genes in 102 species whole genomes at 80% identity and 50% length using CLC genomics Workbench 12 (Qiagen Bioinformatics, CA).

### Example 3

### Bacterial Combination and Formulation of HBT and LBT Consortiums

We used a combinatorial assembly of bacteria from our culture collection to design two mixtures of bacteria from the high bile tolerance (HBT) and low bile tolerance (LBT) groups. We selected nine bacterial species from each of these groups to match the diversity of the gut (4 Bacteroides, 4 Firmicutes and 1 Actinobacteria) (Table 1).

### Example 4

### Static Co-Culture for HBT and LBT Blends Against Clostridium Difficile

Individual bacteria were initially grown to OD600=0.5 and stocked in 10 % DMSO in -80° C. Clostridium difficile R20291 (CD) was used for the inhibition assays and was also grown to OD600=0.5 and stocked in 10% DMSO in -80° C. separately. All the 20 blends formulated were individually mixed with CD in the ratio 9:1 of mBHI broth in deep 96 well plates in triplicates. The co-culture was incubated for 24 hours and 100 µl of the mixture was serially 10-folds diluted in anaerobic PBS and 100 µl was plated onto the C. difficile selective agar (CDSA). The plate was incubated for 24 hours and CD CFUs were counted and compared to CD control and analyzed.

### Example 5

### Inhibition of CD R20291 by HBT10 and LBT10 in a Minibioreactor

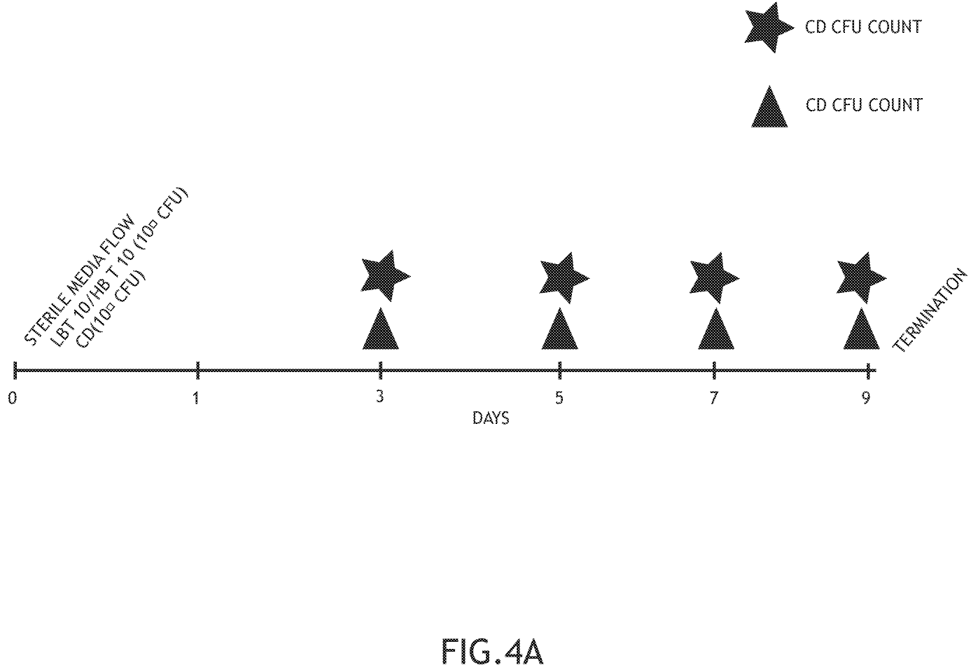

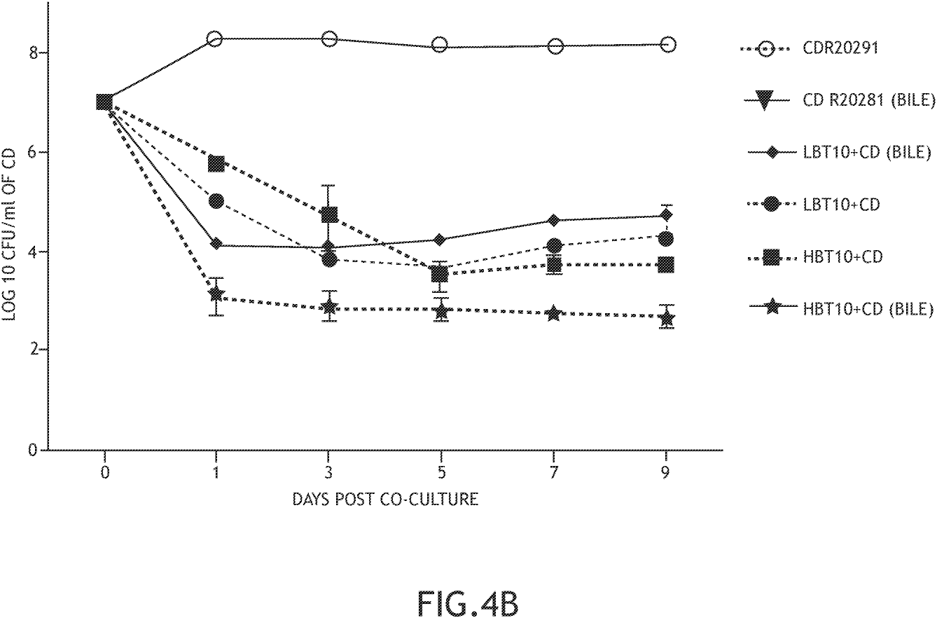

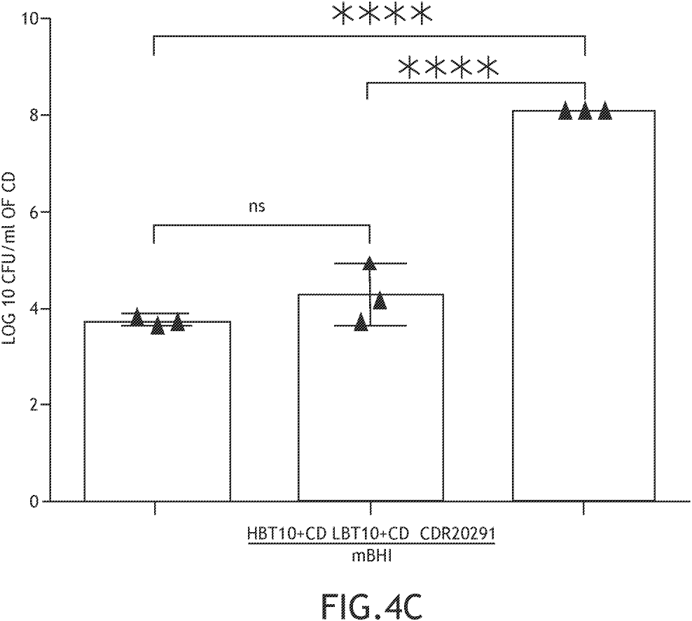

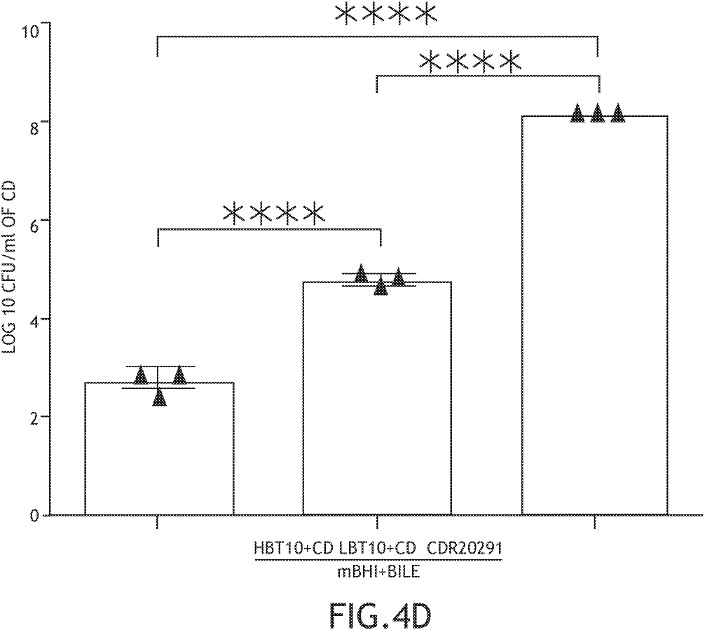

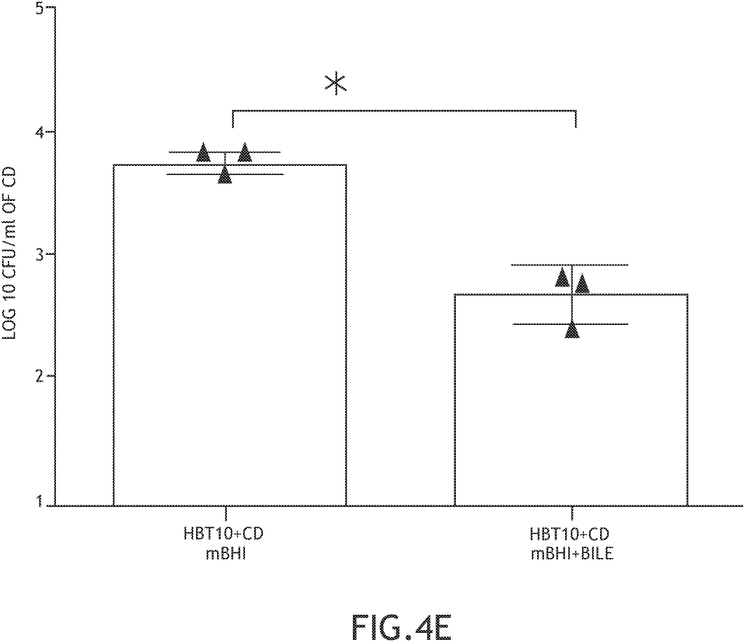

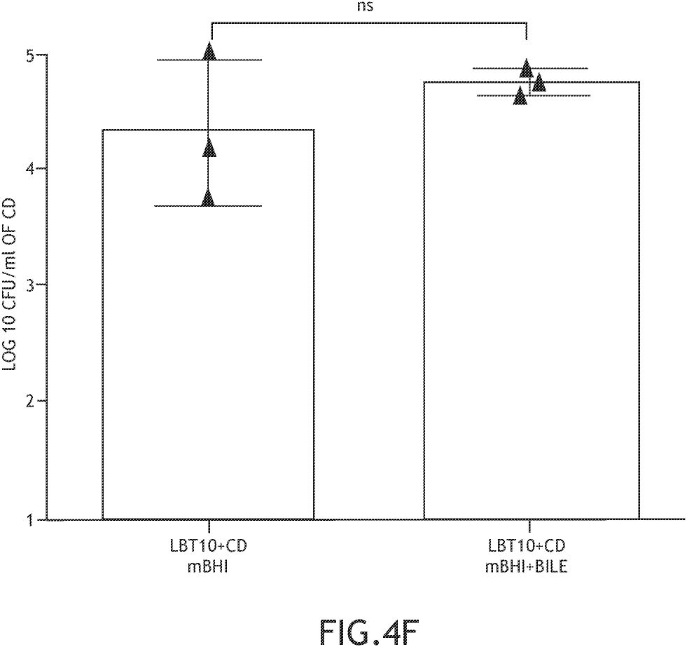

The parent blends HBT10 and LBT10 were tested against CD in minibioreactors as no significant difference between the inhibition status of them within their groups. To determine if LBT10 and HBT10 blends are capable of inhibiting CD in a continuous flow system, these blends were tested in triplicate in minibioreactors following the timeline, as shown in FIG. 4A. The blends were tested in medium with no bile (mBHI) and with 0.5 g/L of bovine bile (to mimic the human gut system) in triplicates. The inlets and outlet pumps were set at 1 and 2 rpm respectively and the magnetic stirrer was set at 130 rpm. The minibioreactor was operated with protocols as described previously with retention time of 12 hours. The assay was performed for 9 days post-cocultures and samples were collected for SCFAs and CD CFU counts at day 0, 1, 3, 5, 7 and 9 post-co-culture (PCC). For CD CFU counts, 100 µl of the sample mixture taken from the bioreactor was taken and 10-folds serially diluted with anaerobic PBS. 50 µl of dilution of 10-1, 10-2, 10-3, 10-4 and 10-5 were plated in CD selective agar and incubated anaerobically for 24 hours. After 24 hours, CD colonies were counted and compared to control CD to determine the inhibition status.

### Example 5

### Inhibition of CD R20291 by HBT10 and LBT10 in Antibiotic-Treated and CD Induced Community in Minibioreactor

CD prevails in the dysbiotic communities after antibiotic treatment. Thus, we created the dysbiotic communities following antibiotic treatment in the minibioreactors and induced CD in both media conditions i.e., mBHI+Bile (9 bioreactors) and mBHI (9 bioreactors) alone. Initially, the media was allowed to flow for sterility check in minibioreactors for 24 hours before inoculation of S7 (pooled S1-S6 fecal samples). 300 µl of mixed fecal sample was inoculated in each bioreactor with retention time of 12 hours. The inlet and outlet pumps were set at 1 and 2 rpm respectively and the magnetic stirrer was set at 130 rpm. The minibioreactor was operated with protocols as described previously. The experiment was performed for 21 days with multiple interventions as shown in the timeline (Timeline). After inoculation of the fecal sample on day 1, the community in each bioreactor was sampled at day 2,3,4 and 5 to ascertain no CD growth. The samples were then plated on CDSA to CD CFUs were counted. 10⁷ CFUs of CD was inoculated at day 5 and CD counts were monitored by plating on CDSA at day 6, 7 and 8 to examine if the community is mature enough to resist invading pathogen.

On day 9, the input media were supplemented with Clindamycin (final concentration in media bottle: 250 µg/mL). The antibiotic was continuously supplied from the input media until day 13 to disrupt the community and make it susceptible to the invasion by CD. To remove the residual antibiotics in the media in minibioreactors, media with antibiotics was replaced with normal mBHI media at day 13 for 24 hours before invading with 10⁷ CFUs of CD once at day 14. CD CFUs were monitored by plating the samples from each minibioreactor at day16 (48 hours after inoculation). The invading CD in those minibioreactors was treated with 10⁷ CFUs of HBT10 or LBT10 or non (CD control) in both mBHI alone and mBHI+bile conditions once daily for 3 days (day 16, 17 and 18). CD CFUs were monitored by plating on CDSA on days 17,18, 19, 20 and 21. Samples were collected on day 9 (Pre-antibiotic), day 14 (Pre CDI) and post HBT10/LBT10 treatment at day 21 for 16S rRNA sequencing and SCFAs analysis.

### Example 6

### DNA Isolation, 16S rRNA Sequencing, and Analysis

Total community DNA from 500 µl of samples was extracted using Powersoil DNA isolation kit (MoBio Laboratories Inc, CA). Briefly, 500 µl of the sample was centrifuged at 10,000×g for 1 minute. The supernatant was removed, and the pellet was added to the tubes with beads. 60 µl of C1 solution was added to the tube and agitated for 10 minutes. The remaining steps were performed according to manufacturer’s instructions. Finally, the DNA was eluted with 30 µl of nuclease-free water. The quality of DNA was measured using NanoDropTM one (Thermo Fisher Scientific, DE) and quantified using Qubit Fluorometer 3.0 (Invitrogen, CA). The samples were stored at -200C until further use.

To analyze the community formed for the CD induced dysbiotic community treated with HBT10 or LBT10, a total of 56 samples (e.g., duplicate samples for inoculum, and 18 each for pre-antibiotic (day 9) post-antibiotic (day 14) and post HBT10/LBT10 treatment (day 21)) were used for the amplicon sequencing using the Illumina MiSeq platform with paired-end V3 chemistry. The library was prepared using Illumina Nextera XT library preparation kit (Illumina Inc, CA) targeting V3 and V4 regions of the 16S rRNA. The libraries were bead normalized and multiplexed before loading into the sequencer.

16S rRNA sequence analysis was performed using Quantitative Insights into Microbial Ecology framework (QIIME, Version 2.0). Briefly, the demultiplexed reads obtained were quality filtered using q2-demux plugin followed by denoising with DADA2. All amplicon sequence variants were aligned with Mafft to construct a phylogeny with fasttree2. The outputs rooted-tree.qza, table.qza, taxonomy.qza were then imported into R for analysis using Phyloseq. Shannon diversity index and Bray-Curtis dissimilarity index were calculated for α- and β-diversity metrics after rarefying the samples to 45,000 reads. T test method was used to analyze the differences in the species richness between the groups. Taxonomy was assigned to ASVs using the q2-feature-classifier classify-sklearn naïve Bayes taxonomy classifier against Greengenes 13_8 99% OTUs reference sequencing. The taxonomy table obtained was used as input to Explicit 2.10.5 for visualization.

### Example 7

### SCFAs Determination

800 µl of samples from each minibioreactor was sampled and mixed with 160 µl of 25% m-phosphoric acid. The mixture was centrifuged at >15,000 rpm for 15 min and the supernatant was collected and frozen at -800C. The samples were thawed and centrifuged at >15,000 rpm for 15 min before collecting 500 µl and loading it to gas chromatography (e.g., Aligent Technologies) for SCFAs analysis.

### Example 8

### Identification of Bile Sensitivity of Strains

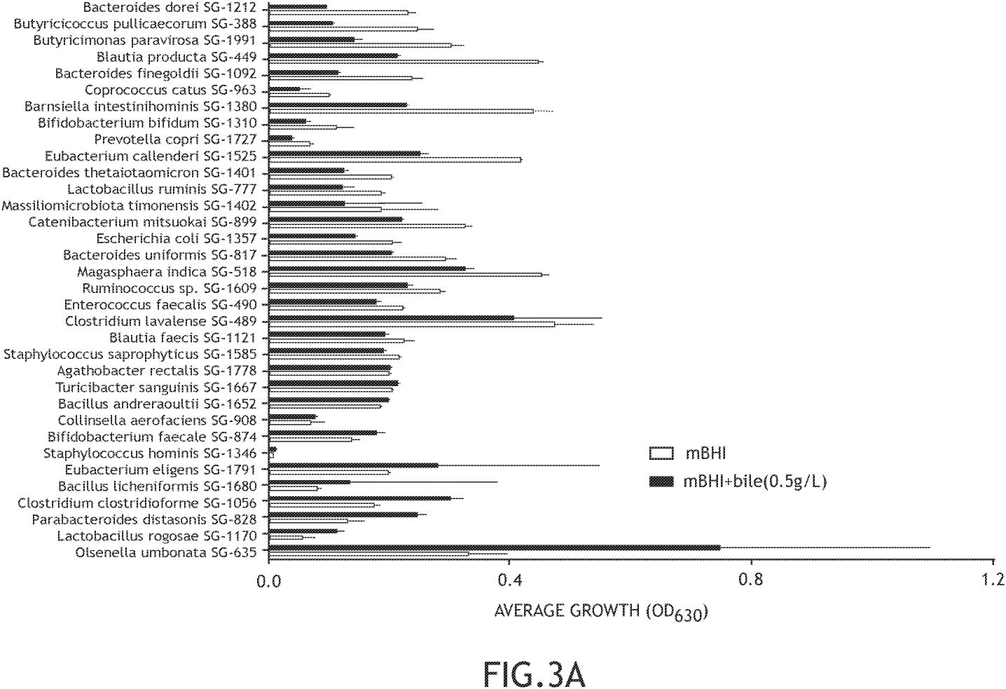

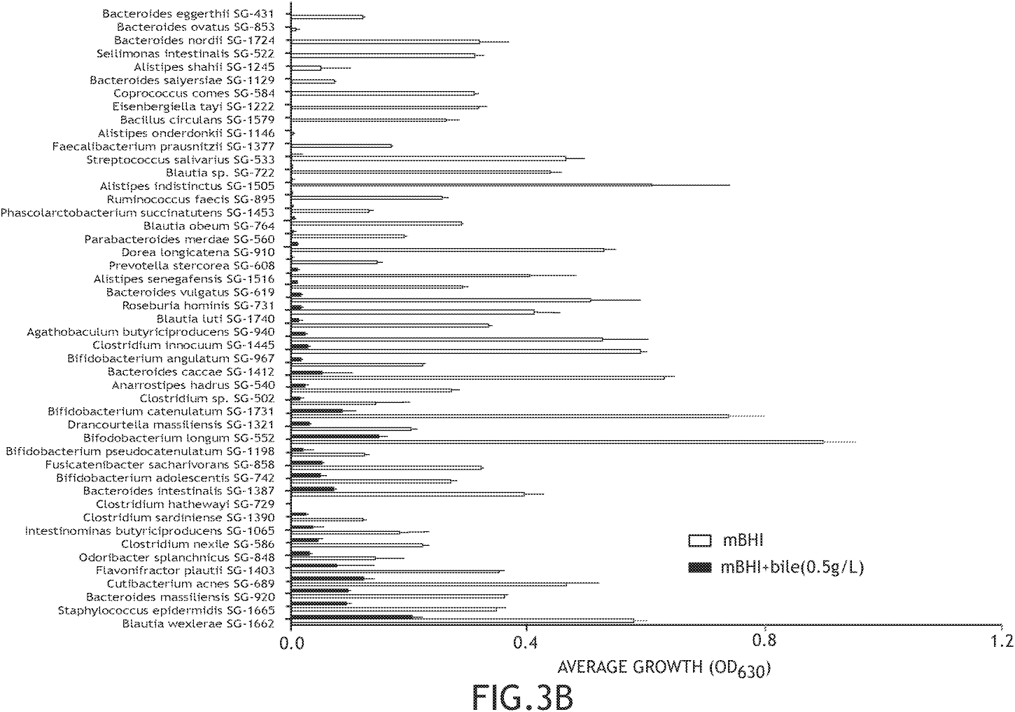

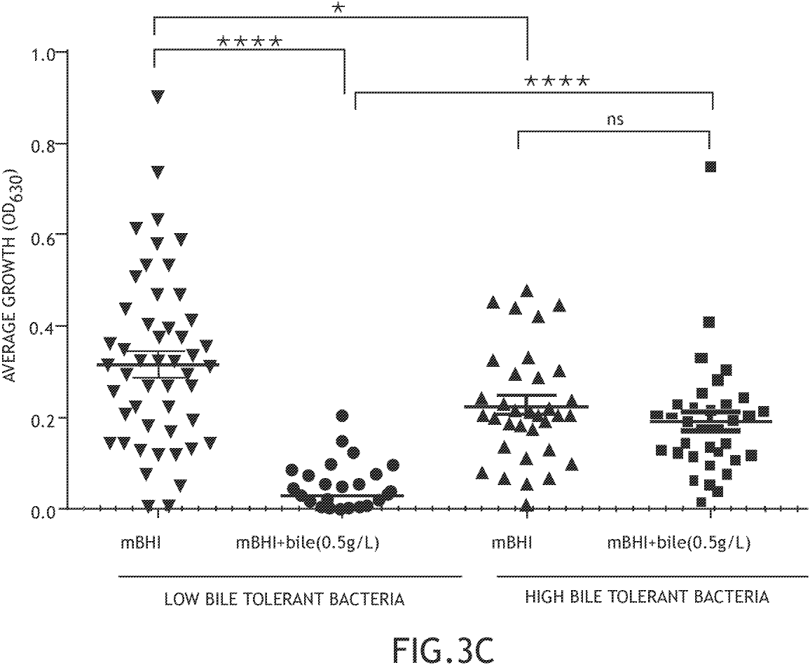

To determine whether any of species in the species library contain genes that convert primary bile acid into secondary bile acid, we searched the genomes for the presence of bai genes in their individual genomes using CLC genomics workbench. None of our species were found to harbor complete operon for the bile conversion. As these bacteria were non-bile converting, we sought to test their ability to tolerate bile. The lower fraction of the cultured bacteria was found to be high bile tolerant, which are listed in FIG. 3A, while higher fraction was found to be low bile tolerant, which are listed in FIG. 3B. Olsenella umbonate, Lactobacillus rogosae, Parabacteroides distasonis, Clostridium clostridioforme, Bacillus licheniformis, and Eubacterium eligens were the top five highest bile tolerant bacteria. Bacteroides eggerthi, Bacteroides ovatus, Bacteroides nordii, Sellimonas intestinalis, and Alistipes shahii were ranked the five lowest bile tolerant bacteria. The low bile tolerant bacterial growth was significantly reduced in addition to bile, but the growth of the high bile tolerant bacterial growth was not affected significantly, as shown in FIG. 3C.

### Example 9

### HBT10 is Highly Efficient in Inhibiting C. Difficile R20291 in Static and Continuous Co-Culture in Vitro

The taxonomic classification of the nine bacteria selected for each of HBT and LBT consortiums are summarized in Table 1. When these blends were tested individually against CD in the ratio 9:1, high bile tolerant consortiums were found to be highly efficient in inhibiting CD compared to the low tolerant groups in mBHI supplemented with bile. The CFU counts of CD alone in both tests with LBT and HBT were found to be non-significant (e.g., Welch two-sample t-test, p=0.218). As the parent blends HBT10 reduced the CD count to log10 0.44±0.44 while LBT10 was not able to inhibit CD with CD counts of log10 7.91±0.08. As both these were distinct from one another in CD inhibition we tested these against CD in continuous flow model in both media conditions (e.g., mBHI and mBHI+bile). Culture compositions and conditions are listed in table 2.

Table 1

Bacterial constituents of high bile tolerant (HBT10) and low bile tolerant (LBT10) consortiums

| Strain | High Bile Tolerant (HBT10) Consortium | Phylum | Family |
|---|---|---|---|
| SG-1727 | Prevotella copri | Bacteroidetes | Prevotellaceae |
| SG-817 | Bacteroides uniformis | Bacteroidetes | Bacteroidaceae |
| SG-1212 | Bacteroides dorei | Bacteroidetes | Bacteroidaceae |
| SG-828 | Parabacteroides distasonis | Bacteroidetes | Porphyromonadaceae |
| SG-1170 | Lactobacillus rogosae | Firmicutes | Lactobacillaceae |
| SG-1791 | Eubacterium eligens | Firmicutes | Lachnospiraceae |
| SG-490 | Enterococcus faecalis | Firmicutes | Lachnospiraceae |
| SG-1680 | Bacillus licheniformis | Firmicutes | Bacillaceae |
| SG-1310 | Bifidobacterium bifidum | Actinobacteria | Bifidobacteriaceae |
| Strain | Low Bile Tolerant (LBT10) Consortium | Phylum | Family |
| SG-608 | Prevotella stercorea | Bacteroidetes | Prevotellaceae |
| SG-431 | Bacteroides eggerthii | Bacteroidetes | Bacteroidaceae |
| SG-619 | Bacteroides vulgatus | Bacteroidetes | Bacteroidaceae |
| SG-560 | Parabacteroides merdae | Bacteroidetes | Porphyromonadaceae |
| SG-910 | Dorea longicatena | Firmicutes | Lachnospiraceae |
| SG-1662 | Blautia wexlerae | Firmicutes | Lachnospiraceae |
| SG-586 | Clostridium nexile | Firmicutes | Lachnospiraceae |
| SG-895 | Ruminococcus faecis | Firmicutes | Lachnospiraceae |
| SG-552 | Bifidobacterium longum | Actinobacteria | Bifidobacteriaceae |

Table 2

Composition of modified Brain Heart Infusion broth (mBHI)

| | |
|---|---|
| Ingredients | per liter |
| Brain heart infusion (BHI) | 37.0 g |
| Yeast extract | 5.0 g |
| L cysteine | 0.3 g |
| Resazurine (0.25 mg/ml solution) | 1 ml |
| Agar | 15.0 g |
| Menadione (5.8 M solution) | 1 ml |
| Vitamin mix ATCC | 10 ml |
| Mineral mix ATCC | 10 ml |
| Hemin (0.5 mg/ml solution)) | 1 ml |
| Acetic acid (30 mM solution) | 1.7 ml |
| Propionic acid (8 mM solution) | 2 ml |
| Isovaleric acid (1 mM solution) | 100 µl |
| Butyric acid (4 mM solution) | 2 ml |

Culture conditions used

| | |
|---|---|
| Culture condition 1 | mBHI alone |
| Culture condition 2 | mBHI + 1 ug/ml Sulphamethoxazole |
| Culture condition 3 | mBHI + 0.5 ug/ml Ciprofloxacin |
| Culture condition 4 | mBHI + 2 ug/ml Erythromycin |
| Culture condition 5 | mBHI + 0.06 ug/mlChlortetracycline |
| Culture condition 6 | mBHI + 2.0 ug/ml Erythromycin + 0.5 ug/ml Ciprofloxacin |
| Culture condition 7 | mBHI + 0.5 ug/ml Imepenem |
| Culture condition 8 | mBHI+ 1 ug/ml Gentamycin |
| Culture condition 9 | mBHI + 0.5 ug/ml Vancomycin |
| Culture condition 10 | mBHI + 1.0 1.0 µg/ml Aztreonam + 10 \.ug/ml Colistin sulphate + 2.0 ug/ml Gentamycin + 0.5 ug/ml Ampicillin + 2.0 ug/ml Erythromycin and 0.25 ug/ml vancomycin |
| Culture condition 11 | heating samples at 70° C. for 15 minutes and plating on mBHI |
| Culture condition 12 | treating sample with 3% chloroform for 1 hour and plating on mBHI |

When LBT10/HBT10 was tested against CD in continuous flow model (FIG. 4A), both were able to inhibit CD significantly in both media conditions (FIG. 4B). The average CD CFU counts for both the blends were reduced from log 7 to 5.06±0.004 and 4.16±0.025 for LBT10 and 5.77±0.135 and 3.09±0.22 for HBT10 in media with and without bile respectively after 24 hours of co-culture. The inhibition of CD by both blends was constant until day 9 where LBT10 reduced it further to 4.09±0.36 and 4.72±0.06 and HBT10 reduced to 3.75±0.046 and 2.67±0.13 in log10 scales in media with and without bile respectively (FIG. 4B). The control CD maintained the high CD counts from day 1 (log10 8.25±0.016) to day 9 (log10 8.14±0.006) and day 1 (log10 8.24±0.011) to day 9 (log10 8.14±0.22) in media with and without bile (FIG. 4B). On day 9, both LBT10 and HBT10 significantly reduced CD counts (p<0.001) in media without bile, as shown in FIG. 4C, but the efficiency of HBT10 was significantly higher compared to LBT10 in media added with bile, as shown in FIG. 4D. Moreover, HBT10 was found to be significantly higher efficient (p= 0.016) in reducing CD loads in media supplemented with bile, as shown in FIG. 4E, while no difference in inhibition levels (p=0.32) was observed for LBT10 when the CD loads were compared in mBHI and mBHI supplemented with bile, as shown in FIG. 4F. Also, on day 9, no significant difference was observed for the CD CFU counts for control CD in both mBHI and mBHI+bile media (e.g., Welch two-sample t-test, p=0.816).

### Example 10

### CD R20291 Was Highly Inhibited by HBT10 in CD Induced Community in Continuous Flow Model in Vitro

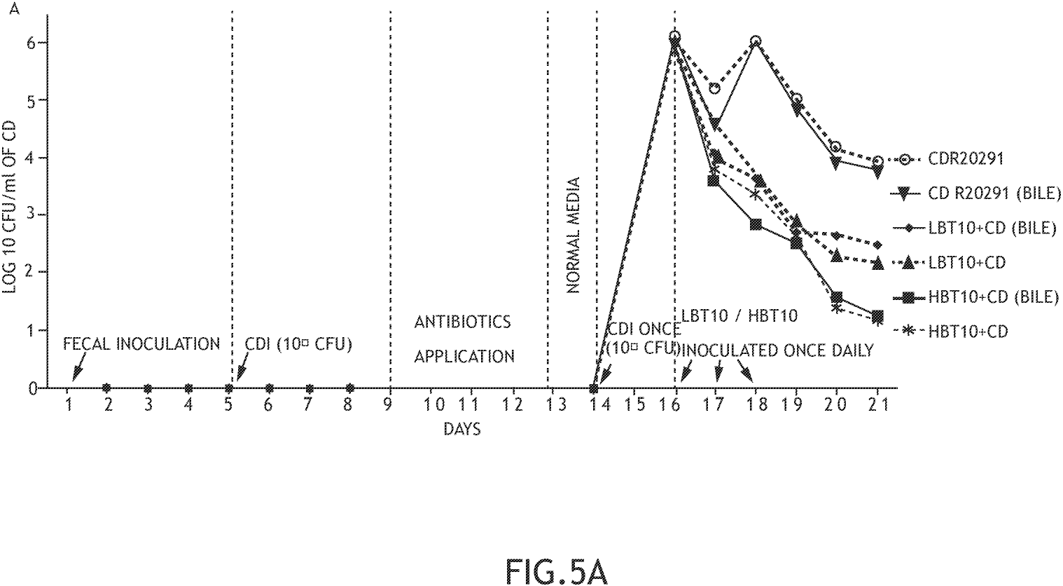

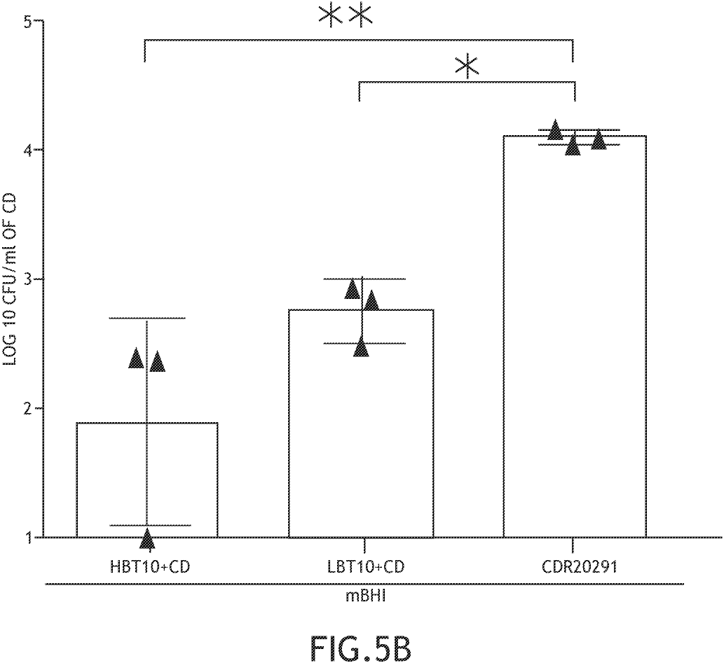

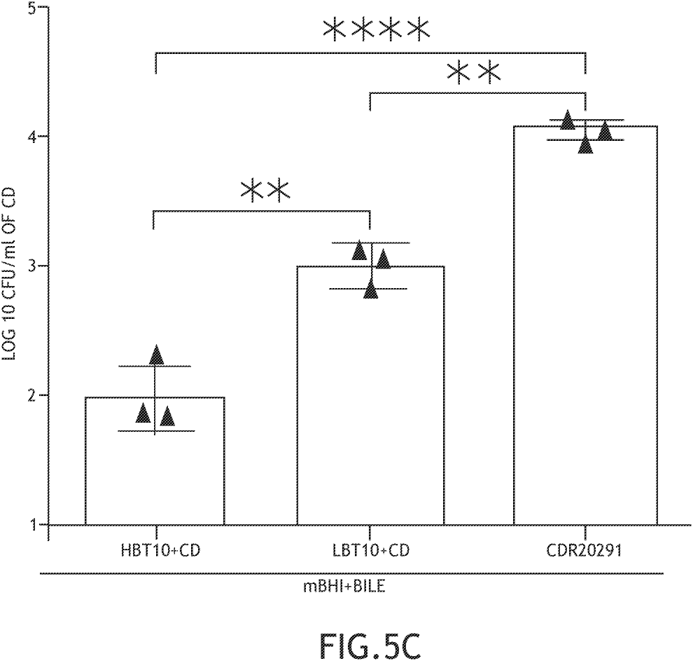

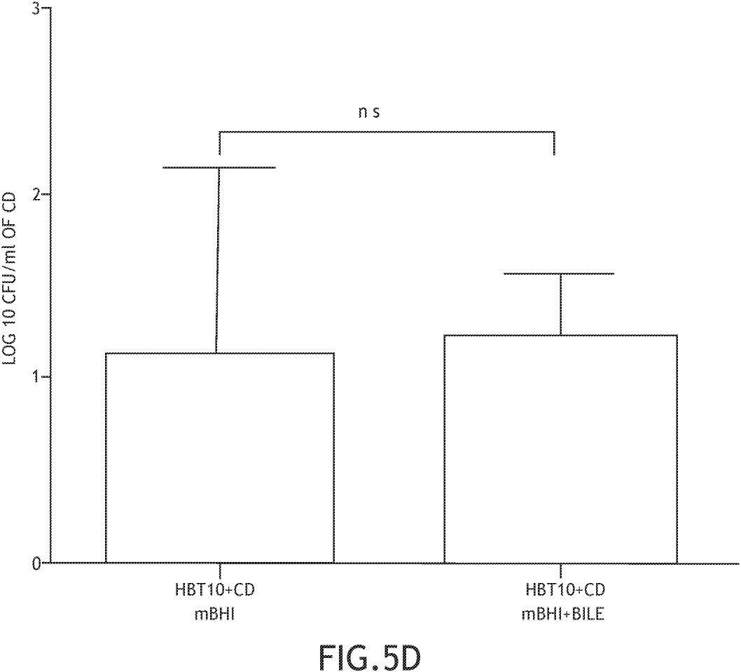

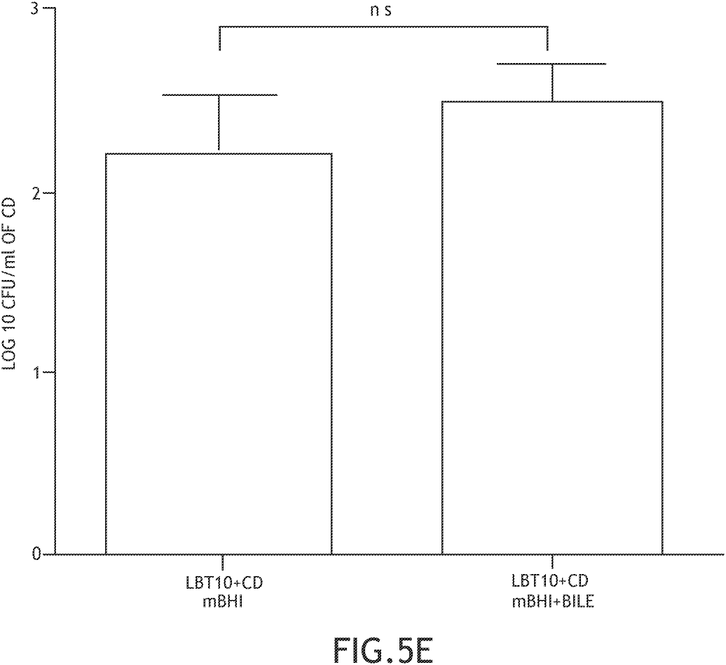

To mimic the human gut system of CDI in humans, we induced CD in each minibioreactor by treating with Clindamycin for four days after 9 days of fecal inoculation. Before antibiotic treatment, at day 5 CD 10⁷ CFUs were inoculated into each minibioreactor to access the ability of the normal microbiota to exclude CD in both mBHI and mBHI supplemented with bile conditions. No colonies CD were observed at day 6, 7 8 and 9 in both conditions following CD inoculation at day 5 implying that normal microbiota formed in both media conditions are mature enough to exclude pathogen, as shown in FIG. 5A. After antibiotic treatment, normal media was allowed to flow for 24 hours to remove residual antibiotics in the medium on day 13. Following, antibiotic treatment, CD 10⁷ CFUs were inoculated in each bioreactors and CD CFUs were counted on day 16, 48 hours after inoculation. CD was able to invade the feces inoculated antibiotic-treated community in both mBHI and mBHI with bile conditions. CD CFU counts in mBHI were 5.95±0.08 and 6.005±0.09 in mBHI with bile which was non-significant to one another on day 16 (Welch two-sample t-test, p=0.64).

Furthermore, this invading CD was treated with 10⁷ CFUs of LBT10/ or none (control) in both mBHI and mBHI+bile in triplicate at day 16, 17 and 18 once daily. CD CFU counts were monitored at days 16, 17, 18. 19, 20 and 21 to access the efficiency of the blends to inhibit CD invasion. CD CFU counts were decreased after treating the CD invaded community in both media conditions on day 17, as shown in FIG. 5A. On day 17, CD CFUs were lowered to log10 4.64±0.11 and log10 3.86±0.13 for the LBT10 and HBT10 respectively in mBHI medium. With addition of bile at day17, CD CFU counts were log10 4.02±0.09 and log10 3.59±0.12 respectively for LBT10 and HBT10 respectively. Interestingly, CD counts for CD control also reduced from average of log10 6.09±0.036 and log10 6.09±0.01 to log10 5.21±0.03 and log10 4.51±0.395 respectively in mBHI and mBHI +bile medium. The levels of inhibition for HBT10 and LBT10 treated continued to lower down until the termination of experiment on day 21, as shown in FIG. 5A. On day 21, CD control maintained an average of log13.91±0.029 and log13.81±0.05 in mBHI and mBHI+bile medium. This CD CFUs count was significantly lower compared to day 16 in mBHI (p<0.001) and mBHI+bile (p<0.001) suggesting that CD invaded community in long run can restore the capabilities to resists CD to some extent. On the other hand, HBT10 and LBT10 both reduced the CD CFU counts lower than CD control at day 21, as shown in FIGS. 5B and 5C. Both HBT10 and LBT10 reduced CD CFU significantly in mBHI medium, but with addition of bile, HBT10 was found to be significantly more efficient in reducing CD number than LBT10, as shown in FIG. 5D. However, at day 21, No significant difference in the CD inhibition was observed for HBT10 and LBT10 in mBHI and mBHI+bile, as shown in FIG. 5E.

### Example 11

### SCFAs Data for HBT10/LBT10 Alone and in Complex Communities

SCFAs play a crucial role to provide direct or indirect colonization resistance against pathogens. Thus, to investigate if there were differences between the treatments in terms of SCFAs production, we analyzed the endpoint data of HBT10/LBT10 alone with CD (e.g., day 9) and CD induced and HBT10/LBT10 treated communities (e.g., day 21). Acetate was the only major SCFA produced when HBT10/LBT10 alone was co-cultured with CD in bioreactors as propionate and butyrate levels were below 1 mM concentration. Interestingly, LBT10 and CD co-cultured together in mBHI+bile medium (e.g., 32.67±6.57 mM) produced significantly higher acetate levels compared to the CD while other groups were not significantly different from the control CD at day 9. But, when these blends were used to treat the CD induced dysbiotic community, at day 21, acetate and propionate were the major detected SCFAs. Even though slight variation was observed, no significant difference in the production of acetate (p=0.143) and propionate (p=0.368) was obtained.

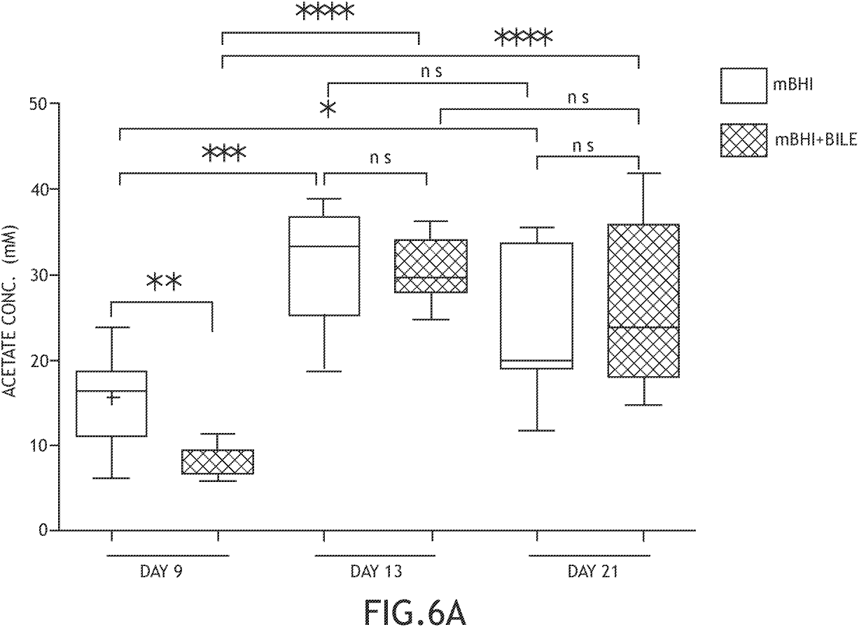

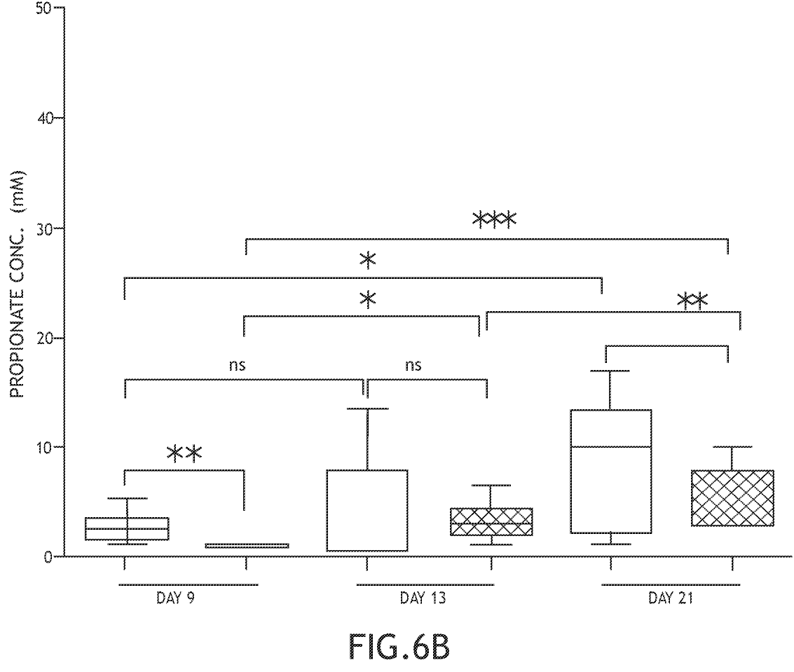

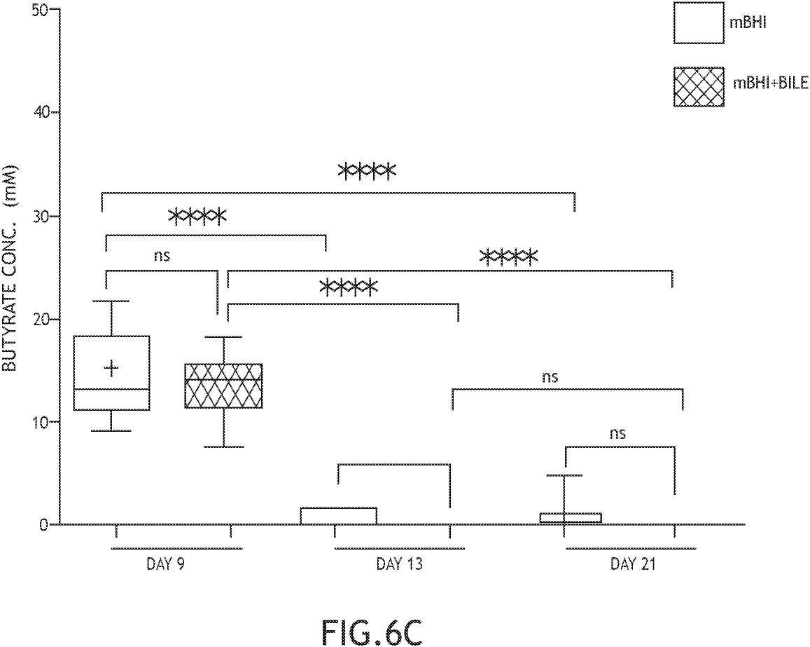

Furthermore, we also explored how the SCFAs vary in the complex community pre- and post-antibiotic treatment and post HBT/LBT10 treatment in the minibioreactors, as shown in FIGS. 6A to 6C. Pre antibiotic treatment (Day9) was dominated by acetate in mBHI medium (15.55±1.86 mM) followed by butyrate (14.85±1.5 mM). Very low amounts of propionate (2.44±0.08 mM) along with less than 1 mM concentrations of isobutyrate, isovalerate and valerate were detected. However, with addition of bile in the medium on day 9, acetate was significantly reduced to 8.18±0.59 mM, as shown in FIG. 6A. Similarly, propionate was significantly reduced to 0.83±0.08 mM. Bile exhibited no significant effect on butyrate production as 13.98±1.04 mM of butyrate was detected in mBHI+bile. However, following antibiotic treatment (e.g., day 13), acetate dominated as the major SCFA. In both mBHI and mBHI+bile media, acetate production significantly augmented to 31.1±2.34 mM and 30.69± 1.26 mM respectively, as shown in FIG. 6A. Such increase acetate came with significant reduction of butyrate to 1.31±0.29 mM and 0.15±0.006 mM respectively for mBHI and mBHI+bile conditions, as shown in FIGS. 4A and 4B. No major changes in the propionate levels were observed even though slight increase in propionate was observed in mBHI+bile condition as the detected amount was below 5 mM concentration, as shown in FIG. 4B. However, the levels of acetate and butyrate were not significantly affected by the additions of HBT10/LBT10 in the medium at end point. A significant increase in propionate levels in mBHI+bile was observed but propionate was still at low concentrations. This suggested that SCFAs provide no major role in CD inhibition for the HBT10 and LBT10 blends in continuous flow model.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

It should be understood that this disclosure is not limited to the particular methodology, protocols, and reagents, etc., described herein and as such may vary. The terminology used herein is for the purpose of describing particular embodiments only, and is not intended to limit the scope of the present disclosure. It is also to be understood that embodiments of the methods disclosed herein may include one or more of the steps described herein. Further, such steps may be carried out in any desired order and two or more of the steps may be carried out simultaneously with one another. Two or more of the steps disclosed herein may be combined in a single step, and in some embodiments, one or more of the steps may be carried out as two or more sub-steps. Further, other steps or sub-steps may be carried in addition to, or as substitutes to one or more of the steps disclosed herein.

A number of embodiments of the invention have been described. Nevertheless, it will be understood that various modifications may be made without departing from the spirit and scope of the disclosure. Accordingly, other embodiments are within the scope of the following claims.

What is claimed:

1. A method for identifying a microbial composition that inhibits colonization of an enteric pathogen in an animal, comprising:
removing a microbial sample from a digestive tract of at least one healthy individual;
culturing the microbial sample;
isolating one or more microbial species within the cultured microbial sample;
identifying the one or more isolated microbial species;
determining one or more bile-tolerant properties of the one or more isolated microbial species;
creating one or more microbial compositions of the one or more isolated microbial species having the one or more bile-tolerant properties;
determining an ability of the one or more microbial compositions to inhibit growth of an enteric pathogen in at least one of an in vitro or an in vivo assay; and
identifying at least one of the one or more microbial composition from the one or more microbial compositions capable of inhibiting growth of enteric pathogens in an animal.

2. The method of claim 1, further comprising determining one or more bile salt conversion properties of the isolated one or more microbial species, wherein the one or more bile salt conversion properties of the one or more isolated microbial species are a determining factor for an addition of the one or more microbial species that are bile-tolerant the microbial composition.

3. The method of claim 1, further comprising: providing antibiotic supplementation to the microbial sample.

4. The method of claim 1, wherein the microbial composition further comprises an antibiotic.

5. The method of claim 1, further comprising administering the microbial composition to the animal to inhibit growth of enteric pathogens.

6. The method of claim 1, wherein the animal is human.

7. The method of claim 1, wherein the enteric pathogen is Clostridioides difficile.

8. A microbial composition that inhibits colonization of an enteric pathogen in an animal, prepared by a process comprising the steps of:
removing a microbial sample from a digestive tract of at least one healthy individual;
culturing the microbial sample;
isolating a microbial species within the cultured microbial sample;
identifying one or more isolated microbial species;
determining one or more bile-tolerant properties of the one or more isolated microbial species;
creating one or more microbial compositions of the one or more isolated microbial species that are bile-tolerant;
determining an ability of the one or more microbial compositions to inhibit growth of an enteric pathogen in at least one of an in vitro or an in vivo assay; and
identifying at least one of the one or more microbial composition capable of inhibiting growth of enteric pathogens in an animal;
fashioning at least one of the one or more microbial composition into a form capable of enteric administration.

9. The microbial composition of claim 8, further including the step of determining one or more bile salt conversion properties of the isolated microbial species, wherein the one or more bile salt conversion properties of the isolated microbial species are a determining factor for an addition of the microbial species that are bile tolerant into the microbial composition.

10. The microbial composition of claim 8, further comprising: providing antibiotic supplementation to the microbial sample.

11. The microbial composition of claim 8, further comprising an antibiotic.

12. The microbial composition of claim 8, wherein the enteric pathogen is Clostridioides difficile.

13. The microbial composition of claim 8, wherein the microbial composition inhibits colonization of an enteric pathogen in a human.

14. The microbial composition of claim 8, wherein the microbial composition includes at least one of Bacteroides, Firmicutes or Actinobacteria strains.

15. The microbial composition of claim 8, wherein the microbial composition comprises at least one of a granule, a pellet, a powder, a liquid, a capsule, or a pill.

16. The microbial composition of claim 8, further comprising at least one of an enteric coating or binder.

17. A method of administering a bile-tolerant microbial composition that inhibits colonization of an enteric pathogen in animals, comprising:
identifying an animal with an at least one of an active enteric disease or risk of enteric disease,
administering to the animal a microbial composition comprised of a mixture of at least one of a bile-tolerant microbial species.

18. The method of claim 17, wherein a selection of one or more microbial species within the bile-tolerant microbial composition is based on an ability of the microbial species to convert bile salts.

19. The method of claim 17, further comprising: providing antibiotic supplementation to the microbial sample.

20. The method of claim 17, wherein the microbial composition further comprises an antibiotic.

21. The method of claim 17, wherein the enteric pathogen is Clostridioides difficile.

22. The method of claim 17, wherein the animal is human.
